(19) United States
(12) Patent Application Publication
(10) Pub. No.: US 2002/0134734 A1
(43) Pub. Date: Sep. 26, 2002

(54) Method for pretreating water for desalination

(75) Inventors: Robert L. Campbell, Rancho Murieta, CA (US); Leo Pedersen, Danville, CA (US)

Correspondence Address: Robert L. Campbell, Ocean Power Corporation, 5000 Robert J. Matthews Parkway, El Dorado, CA, 95762, US

(73) Assignee: Ocean Power Corporation

(21) Appl. No.: 09/941,175

(22) Filed: Aug. 28, 2001

Related U.S. Application Data
(60) Provisional application No. 60/228,826, filed on Aug. 28, 2000.

(51) Int. Cl. B03C 1/00

(52) U.S. Cl. 210/748

(57) ABSTRACT

The present invention provides methods for pretreating water for desalination. According to one embodiment of the invention, a method for pretreating water is provided comprising simultaneously emitting acoustic energy to cause cavitation and light at a wavelength of 200 nm or less at the water. The light causes ozone to be generated. The ozone acts as an oxidizing and anti-foaming agent to sterilize the water. The ozone also inhibits the amalgamation of soft scales in the water. The inventors have also discovered that this method is more efficient than separately applying the acoustic energy and the light to the water. As a result, this method requires on average 25-30% less energy than separately applying the acoustic energy and light. The ozone is preferably removed or destroyed in the water after this method is performed. The ozone may be destroyed by emitting pulsed light at a wavelength of greater than about 200 nm at the water. Another embodiment of the invention is a method for pretreating water for desalination comprising emitting pulsed light at a wavelength of greater than about 200 nm at the water. Typically, the wavelength and intensity of the light is sufficient to destroy ozone in the water.

This application claims the benefit of U.S. Provisional Application No. 60/228,826, filed Aug. 28, 2000, which is hereby incorporated by reference.

### FIELD OF THE INVENTION

This invention relates to methods for pretreating water for desalination with pulsed and continuous ultraviolet light and acoustic energy thereby reducing foaming and fouling tendencies in the desalination process.

### BACKGROUND OF THE INVENTION

In many parts of the world drinking water is not readily available. In fact, the only significant source of water frequently is water containing salt, which has too high a mineral content to meet drinking water standards. Numerous methods and apparatuses have been developed to convert saltwater into fresh (potable) water. Microorganisms, such as bacteria and algae, in the water frequently decrease the performance of such apparatuses and reduce the purity of the fresh water obtained. For example, microorganisms often clog the pipes into and out of a desalination apparatus. Also, scales frequently form on the heated surfaces of an apparatus when it is operated at temperatures over 60° C. See “Automatic Control of Soft Scale Build-up Using Ultrasound”, E. Kishawi and Robert Gampbell, Abu Dhabi Proceedings, Volume III, pages 157-164.

For these reasons, several pretreatment methods have been developed to remove microorganisms and prevent scale formation. For instance, U.S. Pat. No. 4,661,264 discloses a method for disinfecting a fluid. The method involves passing a stream of the fluid through a laser beam which radiates light in the ultraviolet range.

U.S. Pat. No. 5,364,645 discloses a method of controlling microorganisms in food products. The method comprises exposing food to ultraviolet radiation with short high intensity pulses.

There remains a need for improved pretreatment processes for disinfecting and sterilizing saltwater.

### SUMMARY OF THE INVENTION

The present invention provides methods for pretreating water for desalination. The pretreatment methods kill microbes in the water and prevent scale formation during later desalination. According to one embodiment of the invention, a method for pretreating water is provided comprising simultaneously emitting acoustic energy to cause cavitation in the water and light at a wavelength of 200 nm or less at the water. The emitted light purifies the water from microbes and generates ozone in the water, which further enhances the antimicrobial effect of the treatment. The ozone acts as an oxidizing and anti-foaming agent to purify the water. The ozone also inhibits the amalgamation of soft scales in the water. The inventors have also discovered that this method is more efficient than separately applying the acoustic energy and the light to the water. As a result, this method requires on average 25-30% less energy than separately applying the acoustic energy and light. The ozone is preferably removed or destroyed in the water after this method is performed. The ozone may be destroyed by any method known in the art, such as, for example, by emitting continuous or pulsed light at a wavelength of greater than about 200 nm at the water.

Another embodiment of the invention is a method for pretreating water for desalination comprising emitting continuous or pulsed light at a wavelength of greater than about 200 nm at the water. Typically, the wavelength and intensity of the light is sufficient to destroy ozone in the water.

### BRIEF DESCRIPTION OF THE FIGURES

FIG. 1 is a schematic diagram of one exemplary apparatus of the present invention for disinfecting water; and

FIG. 2 is a schematic diagram of another exemplary apparatus of the present invention for disinfecting water.

### DETAILED DESCRIPTION OF THE INVENTION

In any identified embodiments, the term “about” means within 50%, preferably within 25%, and more preferably within 10% of a given value or range. Alternatively, the term “about” means within an acceptable standard error of the mean, when considered by one of ordinary skill in the art.

The present inventors have discovered improved processes for disinfecting water, such as saltwater, and for preventing formation of scales during desalination. One embodiment of the present invention is a method for pretreating water comprising simultaneously emitting acoustic energy of a sufficient intensity to result in an average intensity in the water of from about 1 to about 5 mW/cm³ and light at a wavelength of 200 nm or less at the water.

Generally, the sound pressure volume of the acoustic energy emitted is sufficient to cause cavitation. The acoustic energy is preferably continuously emitted. According to one preferred embodiment, the acoustic energy emitted is sufficient to result in an average intensity in the water of from about 2.5 to about 3.5 mW/cm³ and more preferably about 2.9 or 3.0 mW/cm³.

For many acoustic generators, such as those based on piezoelectric materials, the average intensity of the acoustic energy emitted broadly ranges from about 1 to about 5 Watts/cm². According to one embodiment, the average intensity of the acoustic energy emitted ranges from about 3 to about 4 Watts/cm². According to another embodiment, the average intensity of the acoustic energy emitted is about 3.6 Watts/cm².

According to one embodiment, the audio carrier frequency of the acoustic energy generally ranges from about 800 kHz to about 2 MHz. According to another embodiment, the audio carrier frequency of the acoustic energy generally ranges from about 1600 Hz to about 900 kHz. The audio carrier frequency may be constant or variable. When the audio carrier frequency is varied over time, the rate of change of the audio carrier frequency may be constant or variable. According to one preferred embodiment, the rate of change of the audio carrier frequency is varied sinusoidally over time. For example, the acoustic energy can be pulsed at a frequency of about 840 kHz. Another example is acoustic energy emitted at 1.7 MHz as a continuous sinusoid.

Although the light emitted at a wavelength of 200 nm or less may be pulsed, it is preferably continuous. The wavelength of the light preferably ranges from about 130 to about 190 nm and more preferably ranges from about 170 to about 190 nm. According to one preferred embodiment, the wavelength of the light is about 185 nm. The intensity of the emitted light preferably ranges from about 50,000 to 150,000 Wsec/cm². According to one preferred embodiment, the intensity of the light is about 90,000 Wsec/cm².

The acoustic energy and the light are typically emitted at the water for about 3 to about 20 seconds and preferably for about 5 to about 10 seconds.

The light causes the formation of ozone in the water. The ozone purifies the water by acting as an oxidizing and anti-foaming agent. The ozone also inhibits the amalgamation of soft scales in the water. Since ozone degrades most plastics (such as polypropylene) and various other materials, this method is preferably performed in a container, conduit, or the like (hereinafter collectively referred to as “container”) composed of a material resilient to ozone. Also, the material of the container must permit transmission of light at wavelengths of 200 nm or less and acoustic energy. Suitable materials include, but are not limited to, quartz, aramids, such as Kevlar® available from E. I. du Pont de Nemours and Company of Wilmington, Del., and polyvinylidene fluoride (PVDF), such as Hylar® PVDF available from Ausimont Deutschland GmbH of Bitterfeld, Germany and Kynar® PVDF available from Elf Atochem North America, Inc. of Philadelphia, Pa.

The ozone in the water is preferably removed or destroyed after the acoustic energy and light have been emitted. One method of destroying the ozone in the water is by emitting pulsed light at a wavelength of greater than about 200 nm at the water. Generally, the light has a wavelength and intensity sufficient to destroy ozone in the water.

The wavelength of the light preferably ranges from about 240 to about 280 nM and more preferably ranges from about 260 to about 270 nm. According to one embodiment, the wavelength of the light is about 260 nm.

For germicidal effects, the wavelength is preferably about 270 nm, while for alteration of DNA it is preferably about 254 nm. For ozone destruction, the wavelength of light preferably ranges from about 260 to about 270 nm. Therefore, according to one preferred embodiment, the wavelength of the light covers the spectrum from 250 to 270 nm. Light having a wavelength of less than 200 nm is preferably not emitted at the water, since it may cause formation of ozone.

The power level of the emitted light broadly ranges from about 10,000 to about 100,000 Wsec/cm² and preferably ranges from about 38,000 to about 90,000 Wsec/cm². Ozone at a concentration of about 1 ppm can be destroyed by light having a wavelength of greater than about 200 nm at an intensity of about 90,000 Wsec/cm². Waterborne organisms can be destroyed by light having a wavelength of greater than about 200 nm at an intensity of about 38,000 Wsec/cm². According to a preferred embodiment, the power level of the emitted light is about 80,000 Wsec/cm².

The duration of the pulse broadly ranges from about 0.1 to about 10 milliseconds and preferably ranges from about 0.5 to about 2 milliseconds. Generally, the pulsed light is emitted for a duration sufficient to destroy at least about 90% and preferably at least about 95% by weight of the ozone in the water. The pulsed light is typically emitted at the water for less than about 5 seconds and preferably for about 1 to about 2 seconds. According to one embodiment, the pulse repetition frequency generally ranges from about 1 kHz to about 10 kHz.

According to another embodiment, the water is pretreated by emitting continuous or pulsed light at a wavelength of greater than about 200 nm at the water as discussed above.

The methods of the present invention are preferably performed at less than 60° C. in order to prevent the formation of scales. Preferably, the temperature of the water ranges from about 59 to 77° F., i.e., from about 15 to about 25° C. According to another embodiment, the temperature of the water ranges from about 50 to 59° F., i.e., from about 10 to about 15° C.

After the water has been pretreated, it is preferably desalinated by any method known in the art.

The method of the present invention may be carried out using a number of different types of assemblies in which water flows along a predetermined path while the sound wave and one or more light waves of predetermined wavelengths are emitted at the water. According to one preferred embodiment, the water flows through a conduit, rather than being stagnant, while the sound wave and the one or more light waves are emitted at the water. The flow rate of the water can be selected depending upon the specific application and in view of certain parameters, such as the size and shape of the conduit and the location of light and sound sources. The light and sound sources generate the light and sound waves, respectively, and it will be appreciated that there may be a multiplicity of individual light and sound sources located along the conduit. Preferably, the light and sound sources are stationarily mounted relative to the conduit. The light and sound sources can also be removable and adjustable relative to the conduit.

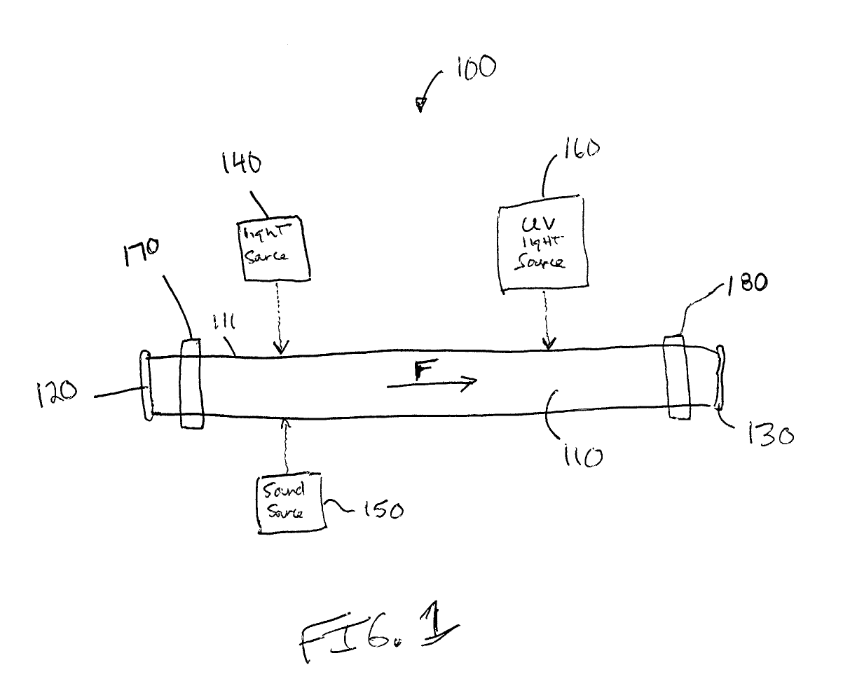

Now referring to FIG. 1 in which one exemplary apparatus for carrying out the present method is shown and generally indicated at 100. The apparatus 100 generally includes a conduit 110 for carrying water according to a predetermined path. The conduit 110 has an inlet section 120 at one end and an outlet section 130 at an opposing second end. While the conduit 110 may be formed of any number of materials, the conduit 110 is preferably formed of a material that is transparent to both ultraviolet light and light at a wavelength of 200 nm or less. In addition, the material forming the conduit 110 offers the desired acoustic characteristics in that the material permits sound waves to travel therethrough and impact the water. More preferably, the conduit is also transparent to light at a wavelength of 200 nm or greater. Suitable materials for the conduit 110 include, but are not limited to, quartz or polymeric materials formed of aramid fibers or polyvinylidene fluoride (PVDF). For example, the conduit 110 maybe formed of a Kevlar® material (commercially available from E. I. du Pont de Nemours and Company of Wilmington, Del.). It has been found that Kevlar® materials offer the desired translucivity of ultraviolet light along with light having a wavelength of 200 nm or less. Furthermore, Kevlar® materials provide excellent mechanical properties which permit the conduit 110 to act as a high pressure fluid conduit, permit sound waves to travel therethrough, and does not degrade when contacted with ozone.

It will be understood that the conduit 110 may have a cross section of varying shapes and sizes and in one exemplary embodiment, the cross section of the conduit 110 is generally circular. Thus, the conduit 110 is generally in the form of an elongated pipe in this one embodiment.

The apparatus 100 further includes at least one light source 140 which is designed to emit light at a wavelength of 200 nm or less at the water. The light source 140 is positioned at a first location relative to the conduit 110 and downstream from the inlet section 120. Preferably, the light source 140 has an orientation such that the light is emitted substantially perpendicular to an outer surface 111 of the conduit 110. Likewise, the light is emitted substantially perpendicular to a flow direction, generally indicated by directional arrow F, of the water within the conduit 110. The light source 140 may comprise any number of light source devices which emit light in the desired wavelength range. For example, the light source 140 may be in the form of one or more lamps.

At the first location of the conduit 110, a sound source 150 is also positioned so that sound emitted therefrom contacts and penetrates the outer surface 111 of the conduit 110. The sound source 150 may comprise any suitable device capable of generating and emitting sound within the desired frequency and intensity ranges previously mentioned. For example, the sound source 150 may comprise one or more acoustic generators which are positioned relative to the conduit 110. The sound source 150 should preferably be located at the first location so that the light source 140 and the sound source 150 simultaneously emit respective waves which contact and treat the water. Thus, FIG. 1 shows the light source 140 and the sound source 150 being spaced apart from one another at the same first location of the conduit 110. It has been found that the above-described advantageous synergistic effect arises when the water is subjected simultaneously to both light (wavelength of 200 nm or less) and sound waves.

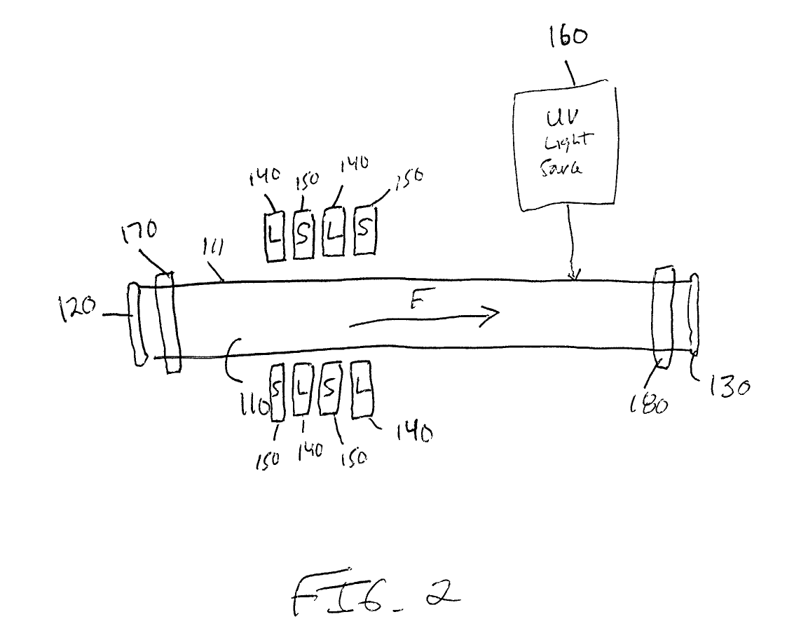

It will be appreciated that the light source 140 and sound source 150 may each comprise a plurality of individual emitting members which are interleaved within one another. This configuration is generally shown in FIG. 2. FIG. 2 shows a plurality of light sources 140 and a plurality of sound sources 150 being arranged in an alternating manner. In this configuration, one light source 140 is spaced apart from one sound source 150 so that at any given location along the conduit 110 where the light sources 140 and sound sources 150 are positioned, the flowing water is simultaneously subjected to both light and sound waves.

It will also be appreciated that the light source 140 and sound source 150 may have a structure which is complementary to the cross section of the conduit 110. For example, when the conduit 110 has an annular cross section, the structures of the light source 140 and sound source 150 may each be generally semi-circular so that the two effectively envelope the conduit 110. Any number of complementary shapes may be selected if the user desires to employ this type of design.

The apparatus 100 further includes at least one ultraviolet light source 160 which is designed to emit ultraviolet light at the water flowing through the conduit 110. The ultraviolet light source 160 typically emits light at a wavelength of greater than about 200 nm and more preferably at from about 240 nm to about 280 nm. The ultraviolet light source 160 is positioned at a second location relative to the conduit 110. The ultraviolet light source 160 is positioned further downstream than the light source 140 and the sound source 150. In other words, the ultraviolet light source 160 is positioned between the first location and the outlet section 130. According to one preferred embodiment, the conduit at the second location completely or substantially blocks (or absorbs) light having a wavelength less than 200 nm from being transmitted therethrough. According to another preferred embodiment, a filter (not shown) for completely or substantially blocking (or absorbing) light having a wavelength less than 200 nm is positioned between the light source 160 and the conduit 110. The filter prevents light having a wavelength of less than 200 nm emitted by the light source 160 from entering the conduit 110.

Preferably, the ultraviolet light source 160 has an orientation such that the ultraviolet light is emitted substantially perpendicular to the outer surface 111 of the conduit 110 and the flow direction F of the water within the conduit 110. The ultraviolet light source 160 may comprise any number of suitable devices and in one exemplary embodiment, the ultraviolet light source 160 comprises one or more ultraviolet lamps.

In one aspect of the present invention, each of the light source 140 and the ultraviolet light source 160 is located external from the conduit 110. In other words, the sources 140, 160 are located away from the conduit 110 and thus are not in contact with the flowing water. By placing the sources 140, 160 away from the water, the water is not heated as is the case where the sources 140, 160 contact the flowing water. In the case where the sources 140, 160 each comprise a lamp type device, a lens cover portion (not shown) of the lamp is in contact with the water and the heating of the water (sea water) causes the formation of a residue (soft scales) on the lamp. This obstructs the emission of the light waves and also adds extra complications because the lamps will require continuous cleaning and maintenance to remove the residue. Because of the transparency of the conduit 110, the sources 140, 160 may preferably be placed externally about the conduit 110 away from contact with the water, while still maintaining the desired effectiveness of the present method. Depending upon the application and the design selection, the sources 140, 160 may be located in close proximity to the outer surface 111 or they may be positioned several inches away or even a greater distance from the outer surface 111. The sound source 150 is also preferably located external to the conduit 110 and is positioned a distance from the conduit 110 which permits the sound waves to travel through the conduit 110 and effectively impact the flowing water. The sound source 150 is preferably positioned so that it does not significantly heat the water in conduit 110.

It will be appreciated that while the light sources 140, 160 and the sound source 150 are preferably located external to the conduit 110 such that they are not in contact with the water, one or more of the light sources 140, 160 and the sound source 150 may be incorporated into the conduit 110.

The apparatus 100 may include a number of conventional components such as pumps and valves for controlling and regulating the flow of the water through the conduit 110. For example, the apparatus 100 shown in FIG. 1 includes a first pump 170 proximate the inlet section 120 and a second pump 180 proximate the outlet section 130.

All patents, applications, articles, publications, and test methods mentioned above are hereby incorporated by reference.

Many variations of the present invention will suggest themselves to those skilled in the art in light of the above detailed description. Such obvious variations are within the full intended scope of the appended claims.

We claim:

1. A method for pretreating water for desalination comprising the step of. simultaneously emitting acoustic energy having a sufficient intensity to result in an average intensity in the water of from about 1 to about 5 mW/cm³ and light at a wavelength of 200 nm or less at the water.

2. The method of claim 1, wherein the acoustic energy is emitted continuously.

3. The method of claim 1, wherein the average intensity in the water of the acoustic energy emitted ranges from about 2.5 to about 3.5 mW/cm³.

4. The method of claim 3, wherein the average intensity in the water of the acoustic energy emitted is about 2.9 mW/cm³ or 3.0 mW/cm³.

5. The method of claim 1, wherein the audio carrier frequency ranges from about 800 kHz to about 2 MHz.

6. The method of claim 1, wherein the sound pressure volume of the acoustic energy emitted is sufficient to cause cavitation.

7. The method of claim 1, wherein the audio carrier frequency is varied over time.

8. The method of claim 7, wherein the rate of change of the audio carrier frequency is varied over time.

9. The method of claim 8, wherein the rate of change of the audio carrier frequency is varied sinusoidally over time.

10. The method of claim 1, wherein the light is emitted continuously.

11. The method of claim 1, wherein the wavelength of the light ranges from about 170 to about 190 nm.

12. The method of claim 1, wherein the water is exposed to the acoustic energy and light for about 3 to about 20 seconds.

13. The method of claim 12, wherein the water is exposed to the acoustic energy and light for about 5 to about 10 seconds.

14. A method for pretreating water for desalination comprising the step of emitting continuous or pulsed light at a wavelength of greater than about 200 nm at the water.

15. The method of claim 14, wherein the wavelength and intensity of the light is sufficient to destroy ozone in the water.

16. The method of claim 14, wherein the wavelength of the light ranges from about 240 to about 280 nm.

17. The method of claim 16, wherein the wavelength of the light is about 260 nm.

18. The method of claim 14, wherein the power level of the emitted light ranges from about 10,000 to about 100,000 Wsec/cm².

19. The method of claim 18, wherein the power level of the emitted light ranges from about 38,000 to about 90,000 Wsec/cm².

20. The method of claim 19, wherein the power level of the emitted light is about 80,000 Wsec/cm².

21. The method of claim 14, wherein the duration of the pulse ranges from about 0.1 to about 10 milliseconds.

22. The method of claim 21, wherein the duration of the pulse ranges from about 0.5 to about 2 milliseconds.

23. The method of claim 14, wherein the pulsed light is emitted at the water for a time sufficient to destroy at least about 90% by weight of the ozone in the water.

24. The method of claim 14, wherein the pulsed light is emitted at the water for less than about 5 seconds.

25. The method of claim 24, wherein the pulsed light is emitted at the water for about 1 to about 2 seconds.

26. A method for pretreating water for desalination comprising the sequential steps of:
(a) simultaneously emitting acoustic energy having a sufficient intensity to result in an average intensity in the water of from about 1 to about 5 mW/cm³ and light at a wavelength of 200 nm or less at the water; and
(b) emitting continuous or pulsed light at a wavelength of greater than about 200 nm at the water.

27. The method of claim 26, wherein the acoustic energy is emitted continuously.

28. The method of claim 26, wherein the average intensity of the acoustic energy emitted ranges from about 2.5 to about 3.5 mW/cm³.

29. The method of claim 28, wherein the average intensity of the acoustic energy emitted is about 2.9 mW/cm³ or 3.0 mW/cm³.

30. The method of claim 26, wherein the audio carrier frequency ranges from about 800 kHz to about 2 MHz.

31. The method of claim 26, wherein the sound pressure volume of the acoustic energy emitted is sufficient to cause cavitation.

32. The method of claim 26, wherein the audio carrier frequency is varied over time.

33. The method of claim 32, wherein the rate of change of the audio carrier frequency is varied over time.

34. The method of claim 33, wherein the rate of change of the audio carrier frequency is varied sinusoidally over time.

35. The method of claim 26, wherein the light in step (a) is emitted continuously.

36. The method of claim 26, wherein the wavelength of the light in step (a) ranges from about 170 to about 190 nm.

37. The method of claim 26, wherein the water is exposed to the acoustic energy and light in step (a) for about 3 to about 20 seconds.

38. The method of claim 37, wherein the water is exposed to the acoustic energy and light in step (a) for about 5 to about 10 seconds.

39. The method of claim 26, wherein the wavelength and intensity of the light in step (b) is sufficient to destroy ozone in the water.

40. The method of claim 26, wherein the wavelength of the light in step (b) ranges from about 240 to about 280 nm.

41. The method of claim 40, wherein the wavelength of the light in step (b) is about 260 nm.

42. The method of claim 26, wherein the power level of the emitted light in step (b) ranges from about 10,000 to about 100,000 Wsec/cm².

43. The method of claim 42, wherein the power level of the emitted light in step (b) ranges from about 38,000 to about 90,000 Wsec/cm².

44. The method of claim 43, wherein the power level of the emitted light in step (b) is about 80,000 Wsec/cm².

45. The method of claim 26, wherein the duration of the pulse in step (b) ranges from about 0.1 to about 10 milliseconds.

46. The method of claim 45, wherein the duration of the pulse in step (b) ranges from about 0.5 to about 2 milliseconds.

47. The method of claim 26, wherein the pulsed light in step (b) is emitted at the water for a time sufficient to destroy at least about 90% by weight of the ozone in the water.

48. The method of claim 26, wherein the pulsed light in step (b) is emitted at the water for less than about 5 seconds.

49. The method of claim 48, wherein the pulsed light in step (b) is emitted at the water for about 1 to about 2 seconds.

50. An apparatus for pretreating water comprising:
(a) a conduit having an inlet and an outlet, the conduit being transparent to ultraviolet light having a wavelength of less than about 200 nm and permitting transmission of acoustic energy therethrough;
(b) an ultraviolet source for emitting light having a wavelength of less than about 200 nm at the conduit; and
(c) an acoustic generator for generating acoustic energy having a sufficient intensity to result in an average intensity in water of from about 1 to about 5 mW/cm³ at the conduit.

51. An apparatus for pretreating water comprising:
(a) a conduit having an inlet and an outlet, the conduit transparent to ultraviolet light having a wavelength of less than about 200 nm and permitting transmission of acoustic energy there through;
(b) a first ultraviolet source for emitting light having a wavelength of less than about 200 nm at a first position along the conduit; and
(c) an acoustic generator for generating acoustic energy having a sufficient intensity to result in an average intensity in the water of from about 1 to about 5 mW/cm³ at the first position along the conduit,
(d) a second ultraviolet source for emitting pulsed ultraviolet light at a second position along the conduit;
wherein the second position is more distal than the first position from the inlet.

52. A method for desalinating water comprising:
(a) pretreating the water by the method of claim 1; and
(b) desalinating the water.

53. A method for desalinating water comprising:
(a) pretreating the water by the method of claim 14; and
(b) desalinating the water.

54. A method for desalinating water comprising:
(a) pretreating the water by the method of claim 26; and
(b) desalinating the water.

55. A method for pretreating water for desalination comprising the step of:
simultaneously emitting acoustic energy at a sufficient intensity to cause cavitation in the water and light at a wavelength of 200 nm or less at the water.
